(19) United States
(12) Patent Application Publication
(10) Pub. No.: US 2022/0218729 A1
(43) Pub. Date: Jul. 14, 2022

(54) USE OF GINSENOSIDE M1 FOR TREATING CANCER

(71) Applicant: Sheau-Long LEE, Taoyuan City (TW)

(72) Inventors: Sheau-Long LEE, Taoyuan City (TW); Yu-Chieh LEE, Taoyuan City (TW); Wan-Han HSU, Taoyuan City (TW)

(73) Assignee: Sheau-Long LEE, Taoyuan City (TW)

(21) Appl. No.: 17/610,951

(22) PCT Filed: Jul. 3, 2020
(86) PCT No.: PCT/CN2020/100060

§ 371 Date: Nov. 12, 2021

Related U.S. Application Data
(60) Provisional application No. 62/870,229, filed on Jul. 3, 2019.

(51) Int. Cl. A61K 31/704; A61P 35/00

(52) U.S. Cl. CPC A61K 31/704; A61P 35/00

(57) ABSTRACT

Using of ginsenoside M1 for treatment in particular cancer, including melanoma and prostate cancer. Specifically, it relates to ginsenoside M1 for use in inhibiting growth and suppressing, reducing, blocking and/or preventing tumor metastasis of melanoma cells and prostate cancer cells.

### RELATED APPLICATIONS

This application claims the benefit of U.S. provisional application No. 62/870,229, filed Jul. 3, 2019, the entire content of which is incorporated herein by reference.

### FIELD OF THE INVENTION

The present invention relates to a new use of ginsenoside M1 for treatment in particular cancer, including melanoma and prostate cancer.

### BACKGROUND OF THE INVENTION

Despite recent advances in cancer treatment, finding an appropriate and effective treatment for some particular cancer still amounts to a difficult and daunting task. Melanoma is a highly aggressive, immunogenic tumor that can modulates the immune system, and a most deadly skin cancer (Liu et al. 2019), which has a higher incidence in the past and is associated with advanced metastasis, advanced diagnosis and advanced chemotherapy tolerance. Unfortunately, current therapies for metastatic melanoma include genetic targeting therapy and a variety of immunotherapy, which still a great deal of difficulty in treatment due to low responsive rates, high toxicity and high cost (Farid Menaa 2013; and White et al. 2009). Therefore, it is urgent to find and develop novel therapeutic strategies for the treatment of melanoma. Prostate cancer is a most common malignancy in men in the worldwide. Prostate cancer generally grows slowly within the prostate gland, it usually with good prognosis by treated with surgery or radiotherapy. However, prostate cancer aggressive may occur, which are capable of metastasizing from the prostate to the other organs, bones and lymph nodes (Thobe et al. 2011 and Datta et al. 2010). Unfortunately, current therapy really provides no chemotherapeutic drugs that are considered high efficiency to treat prostate cancer patients and increases mortality rate when the cancer cells metastasize beyond the gland. Currently effective treatment options for prostate cancer are considerably limited. Therefore, development of novel therapeutic drug will greatly benefit the prostate cancer patients

Ginsenosides, the main active ingredients of ginseng, are known to have a variety of pharmacological activities, e.g. antitumor, antifatique, antiallergic and antioxidant activities. Ginsenosides share a basic structure, composed of gonane steroid nucleus having 17 carbon atoms arranged in four rings. Ginsenosides are metalized in the body, and a number of recent studies suggest that ginsenoside metabolites, rather than naturally occurring ginsenosides, are readily absorbed in the body and act as the active components. Among them, ginsenoside M1, also named Compound K (CK), is known as one metabolite of protopanaxadiol-type ginsenosides via the gypenoside pathway by human gut bacteria. Until now, no prior art references report the effect of ginsenoside M1 in the treatment of the above-mentioned cancer.

### BRIEF SUMMARY OF THE INVENTION

In the present invention, it is unexpected found that ginsenoside M1 is effective in inhibiting growth and migration of particular cancer cells. In the present invention, the cancer is melanoma or prostate cancer.

Therefore, the present invention provides a method for treating the cancer in a subject in need thereof comprising administering an effective amount of ginsenoside M1 to the subject. The present invention also provides use of ginsenoside M1 for manufacturing a medicament for treating the cancer in a subject in need. Further provided is a pharmaceutical composition for use in treating the cancer comprising ginsenoside M1 and a pharmaceutically acceptable carrier.

In particular, the ginsenoside M1 is administered in an amount effective in inhibiting proliferation, inducing apoptosis, decreasing clonogenic activity and/or reducing migration activity of the cancer cells.

In some embodiments, ginsenoside M1 is administered by parenteral or enteral route.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

For the purpose of illustrating the invention, there are shown in the drawings embodiments. It should be understood, however, that the invention is not limited to the preferred embodiments shown. In the drawings:

FIG. 1 shows cell viability of A375.S2 cells treated with ginsenoside M1. Cell viability and cell numbers were counted by Trypan blue exclusion method. The data represent three separate experiments. *p<0.05, ***p<0.005.

FIG. 2 shows colony formation ability of A375.S2 cells treated with ginsenoside M1. The quantitative data represent the number of colonies under the colony formation assay. The data represent three separate experiments. *p<0.05, ***p<0.005.

FIG. 3 shows migration ability of A375.S2 cells treated with ginsenoside M1. The data represent the percent confluency within the wound area under the scratch wound healing assay. The data represent three separate experiments. **p<0.01, ***p<0.005.

FIG. 4 shows apoptosis of A375.S2 cells treated with ginsenoside M1 confirmed by flow cytometry. The data represent percentage of Annexin V/PI double staining positive cells. The data represent three separate experiments. **p<0.01, ***p<0.005.

FIGS. 5A and 5B show apoptosis of A375.S2 cells treated with ginsenoside M1 was confirmed by Western blot analysis. FIG. 5A shows representative Western blots for Pro-Caspase-3, Caspase-3, Pro-Caspase-9, Caspase-9 and Bc1-2 with β-actin as the loading control. FIG. 5B shows semi-quantitative analysis. The data represent three separate experiments. *p<0.05, **p<0.01, ***p<0.005.

FIG. 6 shows cell viability of PC-3 cells treated with ginsenoside M1. Cell viability and cell numbers were counted by Trypan blue exclusion method. The data are expressed as the means±SEM for three separate experiments. *p<0.05, **p<0.01. (One-way ANOVA and subsequent Scheffe's test)

FIG. 7 shows colony formation ability of PC-3 cells treated with ginsenoside M1. The quantitative data represent the number of colonies under the colony formation assay. The data are expressed as the means±SEM for three separate experiments. *p<0.05, ***p<0.005. (One-way ANOVA and subsequent Scheffe's test).

FIG. 8 shows migration ability of PC-3 cells treated with ginsenoside M1. The data represent the percent confluency within the wound area under the scratch wound healing assay. The data are expressed as the means±SEM for three separate experiments. *p<0.05, ***p<0.005. (One-way ANOVA and subsequent Scheffe's test).

FIG. 9 shows apoptosis of PC-3 cells treated with ginsenoside M1 confirmed by flow cytometry. The data represent percentage of Annexin V/PI double staining positive cells. The data are expressed as the means±SEM for three separate experiments. ***p<0.005. (One-way ANOVA and subsequent Scheffe's test).

FIGS. 10A and 10B show apoptosis of PC-3 cells treated with ginsenoside M1 was confirmed by Western blot analysis. FIG. 10A shows representative Western blots for Pro-Caspase-3, Pro-Caspase-9, Caspase-9 and Bax/Bc1-2 with β-actin as the loading control. FIG. 10B shows semi-quantitative analysis. The data are expressed as the means±SEM for three separate experiments. ***p<0.005. (One-way ANOVA and subsequent Scheffe's test).

### DETAILED DESCRIPTION OF THE INVENTION

Unless defined otherwise, all technical and scientific terms used herein have the meaning commonly understood by a person skilled in the art to which this invention belongs. As used herein, the following terms have the meanings ascribed to them unless specified otherwise.

The articles “a” and “an” are used herein to refer to one or more than one (i.e., at least one) of the grammatical object of the article. By way of example, “an element” means one element or more than one element.

The term “comprise” or “comprising” is generally used in the sense of include/including which means permitting the presence of one or more features, ingredients or components. The term “comprise” or “comprising” encompasses the term “consists” or “consisting of.”

Ginsenoside M1, also named Compound K (CK), 20-O-β-D-glucopyranosyl-20(S)-protopanaxadiol, is one of saponin metabolites known in the art. The chemical structure of ginsenoside M1 is as follows:

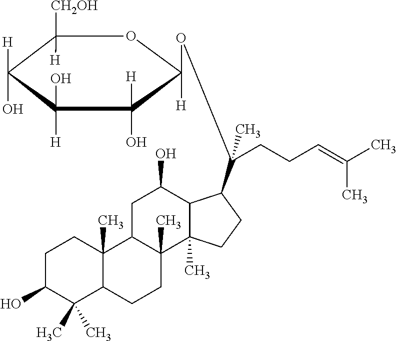

Ginsenoside M1 is known as one metabolite of protopanaxadiol-type ginsenosides via the gypenoside pathway by human gut bacteria. Ginsenoside M1 can be found in blood or urine after intake. Ginsenoside M1 may be prepared from ginseng plants through fungi fermentation by methods known in the art, such as Taiwan Patent Application No. 094116005 (I280982) and U.S. Pat. No. 7,932,057, the entire content of which is incorporated herein by reference. In certain embodiments, the ginseng plants for preparing the ginsenoside M1 include Araliaceae family, Panax genus, e.g. P. ginseng and P. pseudo-ginseng (also named Sanqi). In general, the method of preparation of ginsenoside M1 includes the steps of (a) providing powder of ginseng plant materials (e.g. leaves or stems); (b) providing a fungus for fermenting the ginseng plant materials, wherein the fermentation temperature is ranged from 20-50° C., the fermentation humidity is ranged from 70-100%, the pH value is ranged from 4.0-6.0, and the fermentation period is ranged from 5-15 days; (c) extracting and collecting the fermentation products; and (d) isolating 20-O-β-D-glucopyranosyl-20(S)-protopanaxadiol from the fermentation products.

When ginsenoside M1 is described as “isolated” or “purified” in the present invention, it should be understood as not absolutely isolated or purified, but relatively isolated or purified. For example, purified ginsenoside M1 refers to one that is more purified compared to its naturally existing form. In one embodiment, a preparation comprising purified ginsenoside M1 may comprise ginsenoside M1 in an amount of more than 50%, more than 60%, more than 70%, more than 80%, more than 90%, or 100% (w/w) of the total preparation. It should be understood that when a certain number was used herein to show a ratio or dosage, said number generally includes that within the range of 10% more and less, or more specifically, the scope of 5% more and less than the number.

The term “individual” or “subject” used herein includes human and non-human animals such as companion animals (such as dogs, cats and the like), farm animals (such as cows, sheep, pigs, horses and the like), or laboratory animals (such as rats, mice, guinea pigs and the like).

The term “treating” as used herein refers to the application or administration of a composition including one or more active agents to a subject afflicted with a disorder, a symptom or conditions of the disorder, or a progression of the disorder, with the purpose to cure, heal, alleviate, relieve, alter, remedy, ameliorate, improve, or affect the disorder, the symptoms or conditions of the disorder, the disabilities induced by the disorder, or the progression of the disorder or the symptom or condition thereof. The term “preventing” or “prevention” as used herein refers to application or administration of a composition including one or more active agents to a subject who is susceptible or predisposed to a disorder or a symptom or condition thereof and thus relates to prevention of the occurrence of the disorder or the symptom or condition thereof or underlying causes thereof.

The term “effective amount” used herein refers to the amount of an active ingredient to confer a desired therapeutic effect in a treated subject. For example, an effective amount for treating cancer can be an amount that can prohibit, improve, alleviate, reduce or prevent one or more symptoms or conditions or progression thereof. In some embodiments, an effective amount as used herein can be an amount effective in inhibiting growth, inducing apoptosis, decreasing clonogenic activity and/or reducing migration activity of cancer cells. In some embodiments, for example, treating metastatic melanoma or prostate cancer may include inhibiting or preventing the metastasis of the cancer, a decreasing in the speed and/or number of the metastasis, a reduction in tumor size of the metastasized cancer, a complete or partial remission of the metastasized cancer or any other therapeutic benefit.

In one aspect, according to the present invention, ginsenoside M1 or compositions comprising ginsenoside M1 as the active ingredient may be used in treating individuals with melanoma or at risk of melanoma. The present invention may involve melanoma cancer cells that are premalignant, malignant, metastatic or multidrug-resistant. Accordingly, ginsenoside M1 or compositions comprising ginsenoside M1 as the active ingredient may be administered to individuals with melanoma or individuals at the risk of acquiring melanoma in an amount effective in inhibiting growth and reducing migration of melanoma cancer cells, so as to prevent occurrence of the disease or improve the symptoms or delay deterioration of the symptoms. The treatment of melanoma may comprise inhibition of tumourigenesis of melanoma cancer cells, including inducing apoptosis and reducing metastasis of melanoma cancer cells. In some particular embodiments, the present invention involves use of ginsenoside M1 or compositions comprising ginsenoside M1 as the active ingredient for suppressing, reducing, blocking and/or preventing tumor metastasis of melanoma in a subject in need thereof.

In another aspect, according to the present invention, ginsenoside M1 or compositions comprising ginsenoside M1 as the active ingredient may be used in treating individuals with prostate or at risk of prostate cancer. The prostate cancer as described herein can be benign, malignant, or metastatic. The prostate cancer as described herein can be androgen-insensitive, hormone-resistant, or castrate-resistant. Accordingly, ginsenoside M1 or compositions comprising ginsenoside M1 as the active ingredient may be administered to individuals with melanoma or individuals at the risk of acquiring prostate cancer in an amount effective in inhibiting growth and reducing migration of prostate cancer cells, so as to prevent occurrence of the disease or improve the symptoms or delay deterioration of the symptoms. The treatment of prostate cancer may comprise inhibition of tumourigenesis of prostate cancer cells, including inducing apoptosis and reducing metastasis of prostate cancer cells. In some particular embodiments, the present invention involves use of ginsenoside M1 or compositions comprising ginsenoside M1 as the active ingredient for suppressing, reducing, blocking and/or preventing tumor metastasis of prostate cancer in a subject in need thereof.

The therapeutically effective amount may change depending on various reasons, such as administration route and frequency, body weight and species of the individual receiving said pharmaceutical, and purpose of administration. Persons skilled in the art may determine the dosage in each case based on the disclosure herein, established methods, and their own experience. For example, in certain embodiments, the oral dosage of ginsenoside M1 used in the present invention is 10 to 1,000 mg/kg daily. In some examples, the oral dosage of ginsenoside M1 used in the present invention is 100 to 300 mg/kg daily, 50 to 150 mg/kg daily, 25 to 100 mg/kg daily, 10 to 50 mg/kg daily, or 5 to 30 mg/kg daily. In addition, in some embodiments of the invention, ginsenoside M1 is administered periodically for a certain period of time, for example, daily administration for at least 15 days, one month or two months or longer.

In some embodiments, a therapeutically effective amount of the active ingredient may be formulated with a pharmaceutically acceptable carrier into a pharmaceutical composition of an appropriate form for the purpose of delivery and absorption. Depending on the mode of administration, the pharmaceutical composition of the present invention preferably comprises about 0.1% by weight to about 100% by weight of the active ingredient, wherein the percentage by weight is calculated based on the weight of the whole composition.

As used herein, “pharmaceutically acceptable” means that the carrier is compatible with the active ingredient in the composition, and preferably can stabilize said active ingredient and is safe to the individual receiving the treatment. Said carrier may be a diluent, vehicle, excipient, or matrix to the active ingredient. Some examples of appropriate excipients include lactose, dextrose, sucrose, sorbose, mannose, starch, Arabic gum, calcium phosphate, alginates, tragacanth gum, gelatin, calcium silicate, microcrystalline cellulose, polyvinyl pyrrolidone, cellulose, sterilized water, syrup, and methylcellulose. The composition may additionally comprise lubricants, such as talc, magnesium stearate, and mineral oil; wetting agents; emulsifying and suspending agents; preservatives, such as methyl and propyl hydroxybenzoates; sweeteners; and flavoring agents. The composition of the present invention can provide the effect of rapid, continued, or delayed release of the active ingredient after administration to the patient.

According to the present invention, the form of said composition may be tablets, pills, powder, lozenges, packets, troches, elixers, suspensions, lotions, solutions, syrups, soft and hard gelatin capsules, suppositories, sterilized injection fluid, and packaged powder.

The composition of the present invention may be delivered via any physiologically acceptable route, such as oral, parenteral (such as intramuscular, intravenous, subcutaneous, and intraperitoneal), transdermal, suppository, and intranasal methods. Regarding parenteral administration, it is preferably used in the form of a sterile water solution, which may comprise other substances, such as salts or glucose sufficient to make the solution isotonic to blood. The water solution may be appropriately buffered (preferably with a pH value of 3 to 9) as needed. Preparation of an appropriate parenteral composition under sterile conditions may be accomplished with standard pharmacological techniques well known to persons skilled in the art, and no extra creative labor is required.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### Examples

Ginsenoside M1, also named Compound K (CK), 20-O-β-D-glucopyranosyl-20(S)-protopanaxadiol (named M1 below), was prepared by methods known in the art, such as those described in Taiwan Patent Application No. 094116005 (1280982) and U.S. Pat. No. 7,932,057.

In the present study we have investigated the efficacy and associated mechanisms of M1 against particular cancer, including melanoma and prostate cancer.

Malignant melanoma is a highly aggressive most deadly skin cancer. Unfortunately, current therapies for metastatic melanoma still difficulty in treatment due to high toxicity and side effects. Therefore, novel and effective treatments are needed to control malignant melanoma. In the present study we have investigated the anti-tumor efficacy and associated mechanisms of ginsenoside M1. Our results show that ginsenoside M1 dose-dependently inhibited the cell viability, colony formation and migration ability of human malignant melanoma A375.S2 cells. To further investigate the mechanism of action of ginsenoside M1, we preformed targeting hallmarks of apoptosis by Annexin V/PI double staining by flow cytometry and apoptosis associate protein expression by Western blot analysis. The data show that ginsenoside M1 significantly increased Annexin V/PI double positive staining and activation of caspase-3 and caspase-9 in A375.S2 cells. These results indicate that ginsenoside M1 may be a potential therapeutic against malignant melanoma

Prostate cancer is a most common malignancy in men. However, current therapy really no chemotherapeutic drugs that are considered high efficiency to treat metastasize prostate cancer patients. Ginsenoside M1 has therapeutic effects on several cancer models has been reported, but its therapeutic effect on prostatic remains largely unknown. In the present study, to investigates apoptotic effect of ginsenoside M1 on prostate cancer and the mechanism of action. The results demonstrated that ginsenoside M1 inhibits the cell viability, colony formation and migration ability of human prostate cancer PC-3 cells. To further investigate mechanisms underlying the apoptosis-inducing activity of ginsenoside M1. We found that ginsenoside M1 administration induce Annexin V/PI double staining and apoptosis-related protein expression in PC-3 cells. These results indicate that ginsenoside M1 may be potential and effective therapeutic drug on prostate cancer.

1. Materials and Methods

1.1 Cell Culture

Human malignant melanoma A375.S2 cell line was obtained from the American Type Culture Collection (ATCC, USA). A375.S2 cells were grown in minimum essential medium supplemented with 10% fetal bovine serum, 100 U/mL of penicillin, and 100 μg/mL of streptomycin (Life Technologies, USA). PC-3 cells (hormone-independent human prostate cancer cells) were cultured in Ham's F12K medium supplemented with 7% fetal bovine serum, 100 U/mL of penicillin, and 100 μg/mL of streptomycin (Life Technologies, USA). Cells were cultured at 37° C. in a 5% CO₂ incubator.

1.2 Cell Viability

Seeding cells (2.5×10⁵ cells/well) in 6-well plates and incubated with different concentrations of M1 (10, 20 and 40 μM), cisplatin (50 μM) or vehicle for 24 h. Cells were detached by trypsinization and the cell numbers were counted by trypan blue exclusion method.

1.3 Colony Formation Assay

Plate 500 cells per well into 6-well plates and incubated with different M1 concentrations (10, 20, and 40 μM) or vehicle for 10 days. Cells were fixed in 4% paraformaldehyde after washing with cold PBS twice, and stained with 0.5% crystal violet 10 min at room temperature.

1.4 Wound Healing Assay

Cells were seeded at 6×10⁵ into 6-well plates and incubated for 24 h. Then used a sterile 200-μl pipette tip to create a straight gap of constant width, and each well was washed with sterile PBS. Next, the cells were incubated with various concentrations of M1 (10, 20, and 40 μM) or vehicle. The cells were photographed at 0 and 24 h under a microscope at 100× magnification.

1.5 Flow Cytometry Analysis

A375.S2 cells were seeded at 2.5×10⁵ into 6-well plates, and incubated with M1 (40 μM), cisplatin (50 μM) or vehicle for 24 h. To perform apoptosis using an Annexin V-FITC/PI apoptosis detection kit (BD Biosciences, USA) according to the manufacturer's instructions, and then analyzed by flow cytometry (BD Biosciences).

1.6 Western Blot Analysis

Plate 4×10⁵ cells per well into 6-cm dish, and incubated with different concentrations of M1 (10, 20 and 40 μM), cisplatin (50 μM) or vehicle for 24 h. The cells were exacted by disruption in RIPA buffer (Sigma-Aldrich, Germany) in the presence of protease inhibitor cocktail. Each protein sample was run on a 12% SDS-PAGE gel and transferred to PVDF membrane, which was then incubated for 1 h at room temperature in blocking buffer and then overnight at 4° C. with primary antibodies against caspase-3, caspase-9 (Cell Signaling Technology, USA), Bax, Bcl-2 or β-actin (Santa Cruz, USA). The membrane was incubated for 1 h at room temperature with HRP-conjugated secondary antibodies. The proteins were detected using enhanced chemiluminescence reagent (GE, UK).

1.7 Statistical Analysis

Values are means±SE. Comparison between two groups was performed using Student's t-test. P value <0.05 was considered statistically significant.

2. Results

2.1 Melanoma

2.1.1 M1 Decreased the Viability of Melanoma Cells

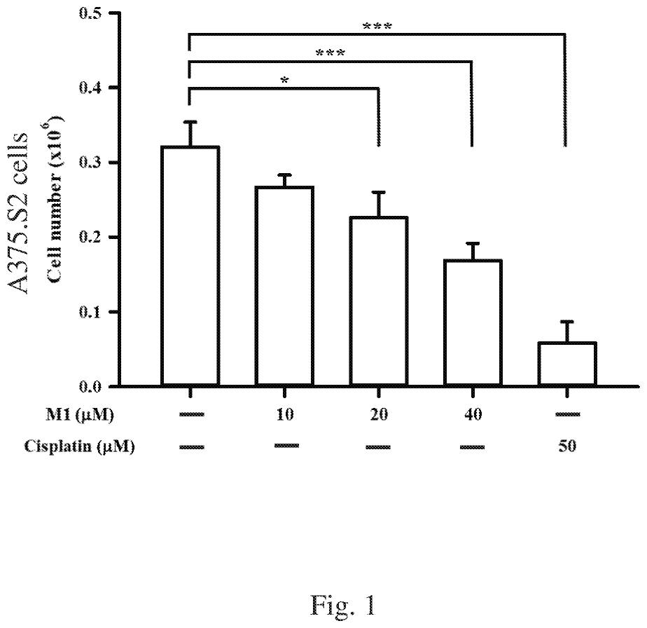

To investigates the effect of M1 on the inhibition of human malignant melanoma cell growth, A375 cells were cultured with various concentration of M1. As shown in FIG. 1, M1 dose-dependently and cisplatin, as a positive control, decreased the cell numbers of A375.S2 cells compared to the control. The data showed that M1 significantly inhibited the viability of human malignant melanoma cells.

2.1.2 M1 Reduced the Colony Formation Ability of Melanoma Cells

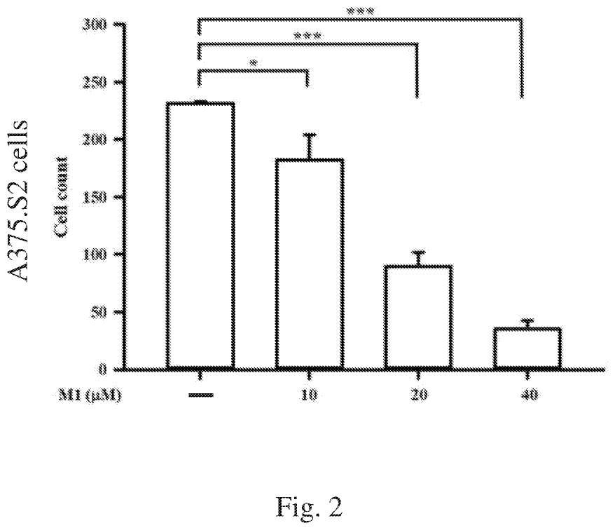

To investigates the effect of M1 on the reduction of colony formation ability of melanoma cells, A375. S2 cells were subjected to colony formation assay with various concentration of M1. As shown in FIG. 2, the numbers of colonies of A375.S2 cells were reduced dose-dependently. The data showed that M1 effectively reduced the colony formation ability of melanoma cells.

2.1.3 M1 Inhibited Migration Ability of Melanoma Cells

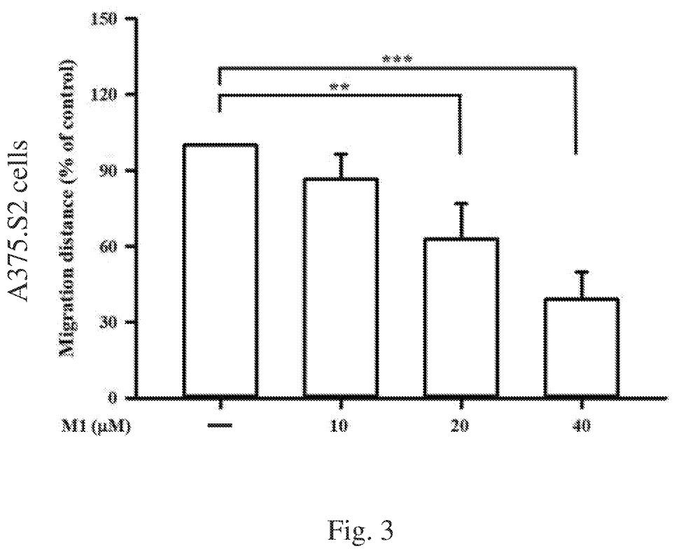

To investigates the effect of M1 on the inhibition of migration ability of melanoma cells, A375.S2 cells were subjected to the wound healing assay with various concentration of M1. As shown in FIG. 3, M1 at 20 or 40 μM for 24 h markedly decreased the cell migration ability compare to control. The data showed that M1 effectively inhibited the migration ability of melanoma cells.

2.1.4 M1 Induced Apoptosis f Melanoma Cells

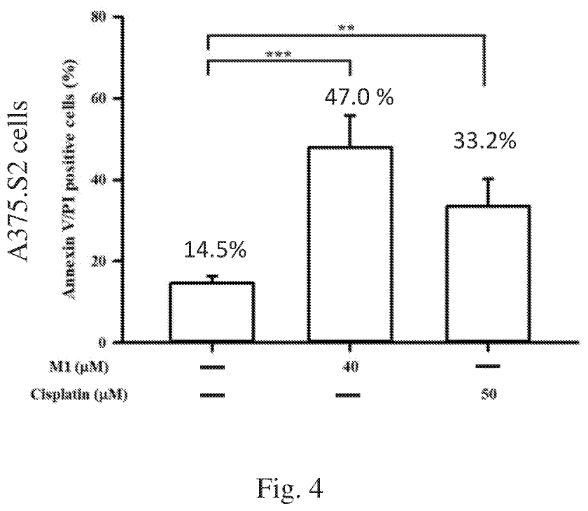

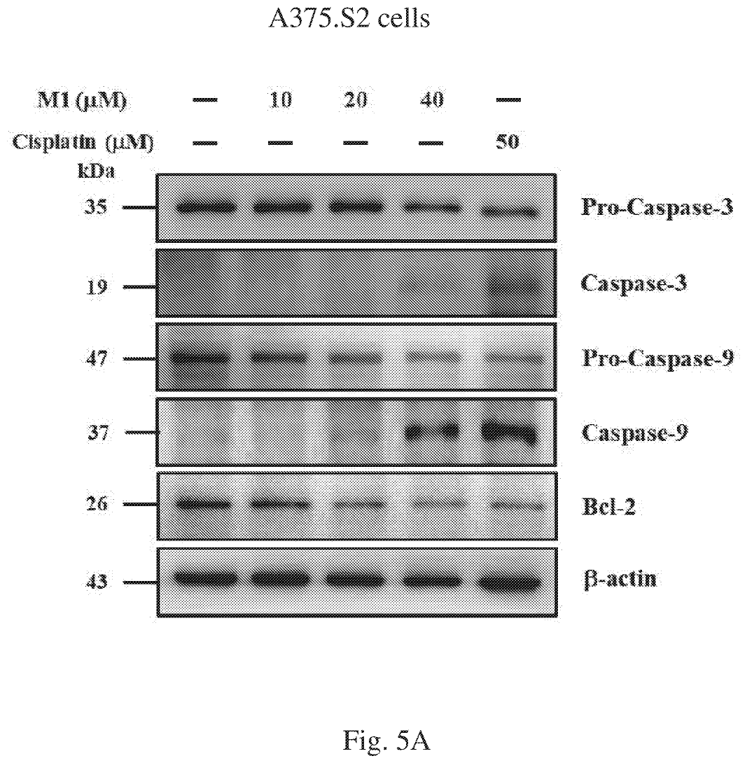

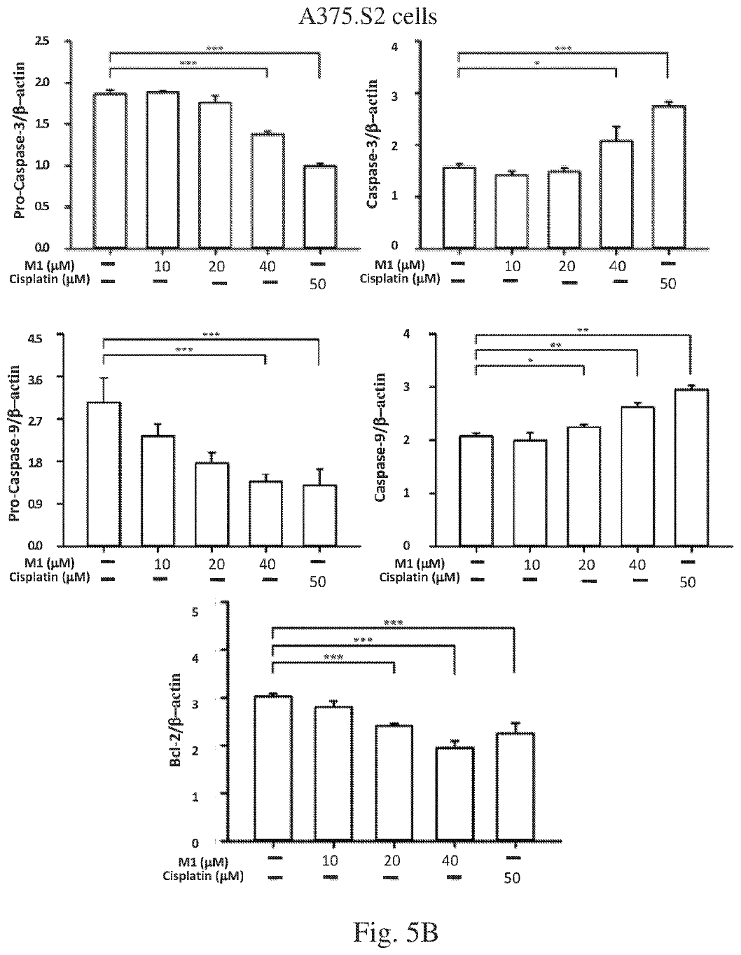

To further investigate mechanism of action of M1, we used Annexin V/PI double staining by flow cytometry and crucial protein expression by Western blot analysis to measured targeting hallmarks of apoptosis. As shown in FIG. 4, 40 μM of M1 and 50 μM of cisplatin for 24 h administration greatly increased the percentage of A375.S2 cells with Annexin V/PI double staining. Moreover, we found that M1 decrease pro-caspase-3, pro-caspase-9 and Bcl-2 protein expression in a dose-dependent manner, while levels of caspase-3 and caspase-9 were significantly increased in A375.S2 cells (FIGS. 5A and 5B). These results indicated that M1 activated apoptosis in human malignant melanoma cells.

2.2 Prostatic Cancer

2.2.1 M1 Inhibits Human Prostatic Cancer Cell Viability

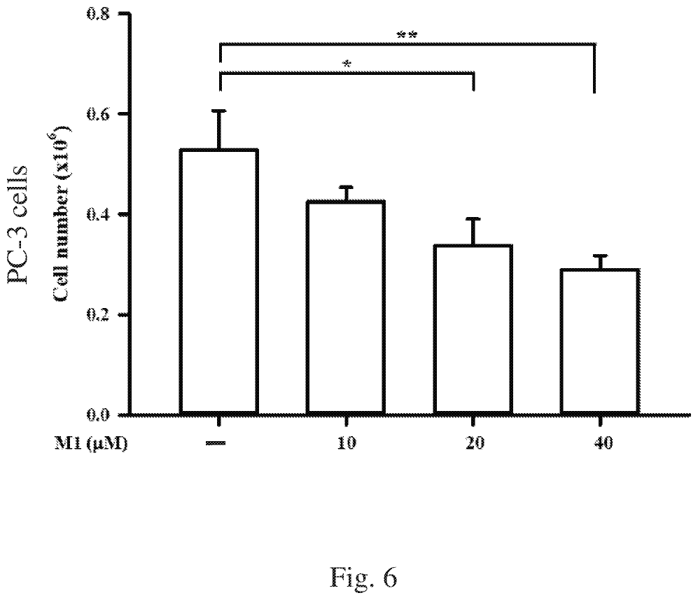

Plate human prostatic cancer cell line PC-3 2.5×10⁵ per 6-well plates and cultured in Ham's F12K medium supplemented with 7% fetal bovine serum, 100 U/mL of penicillin, and 100 μg/mL of streptomycin (Life Technologies, USA). Cells were cultured overnight at 37° C. in a 5% CO₂ incubator, and then treated with different concentrations of M1 (10, 20 and 40 μM) or vehicle for 24 h, and the cell numbers were counted by trypan blue exclusion method. Our result shows that M1 significantly decrease cell numbers of PC-3 cells in dose-dependent manner (FIG. 6). The result indicates that M1 inhibited human prostatic cancer cell viability. The data are expressed as the means±SEM for three separate experiments. *p<0.05, **p<0.01. (One-way ANOVA and subsequent Scheffe's test).

2.2.2 M1 Decreases the Colony Formation Ability in PC-3 Cell

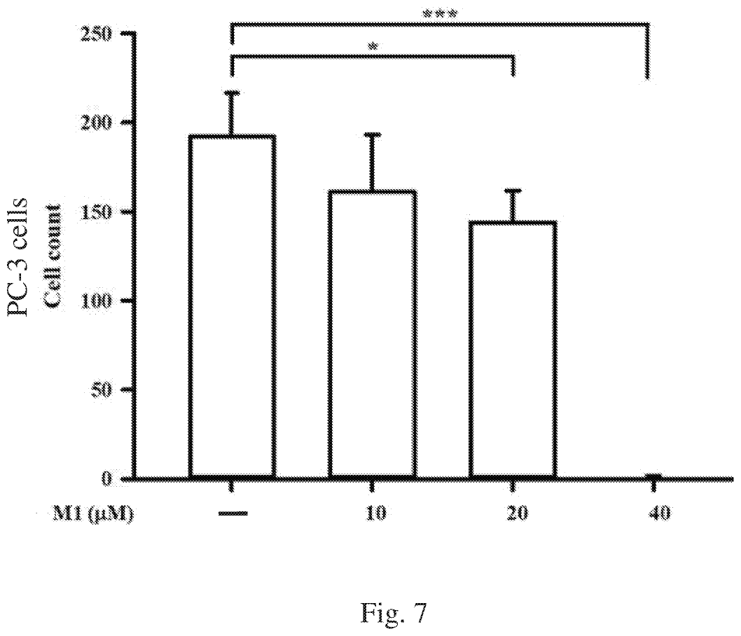

Seeding 500 cells per 6-well plates and treated for 10 days with different concentrations of M1 (10, 20, and 40 μM) or vehicle. The cells were fixed in 4% paraformaldehyde for 15 min, after washing with cold PBS twice, and stained with 0.5% crystal violet 10 min at room temperature. As shown in FIG. 7, we found that M1 inhibited the colony formation ability of PC-3 cells in dose-dependent manner compare to vehicle. The data are expressed as the means±SEM for three separate experiments. *p<0.05, ***p<0.005. (One-way ANOVA and subsequent Scheffe's test).

2.2.3 M1 Reduces the Migration Ability in PC-3 Cell

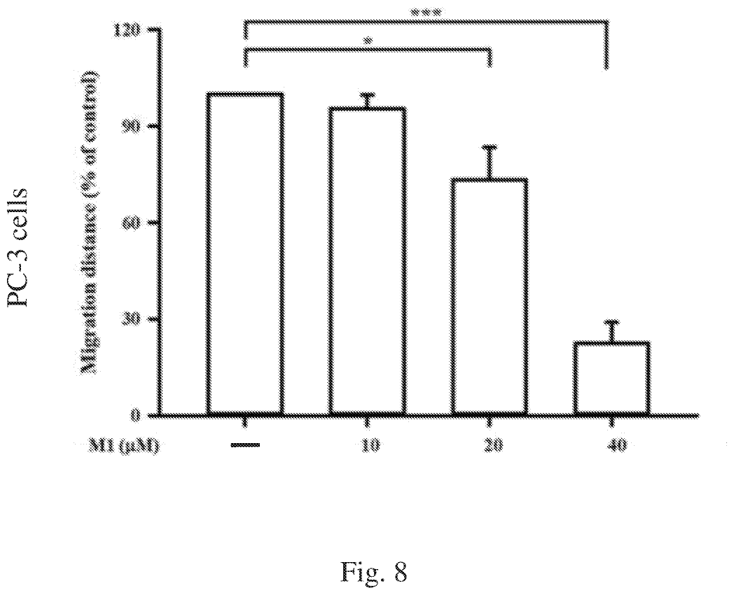

Next, we performed whether M1 reduces the migration ability by wound healing assay in PC-3 cells. Cells were seeded at 6×10⁵ per 6-well plates in culture medium and were grown overnight at 37° C. in a 5% CO₂ incubator. Each well was scratched with sterile 200-μl pipette tip in the cell monolayer, and washed with sterile PBS twice. The cells were treated with M1 (10, 20, and 40 μM) or vehicle for 24 h. The wounds photographed at 0 and 24 h under a microscope at 100× magnification. We observed M1 (20 or 40 μM)-treated PC-3 cells were significantly decreased the cell migration ability compare to vehicle (FIG. 8). The data are expressed as the means±SEM for three separate experiments. *p<0.05, ***p<0.005. (One-way ANOVA and subsequent Scheffe's test).

2.2.4 M1 Reduces Apoptosis in PC-3 Cell

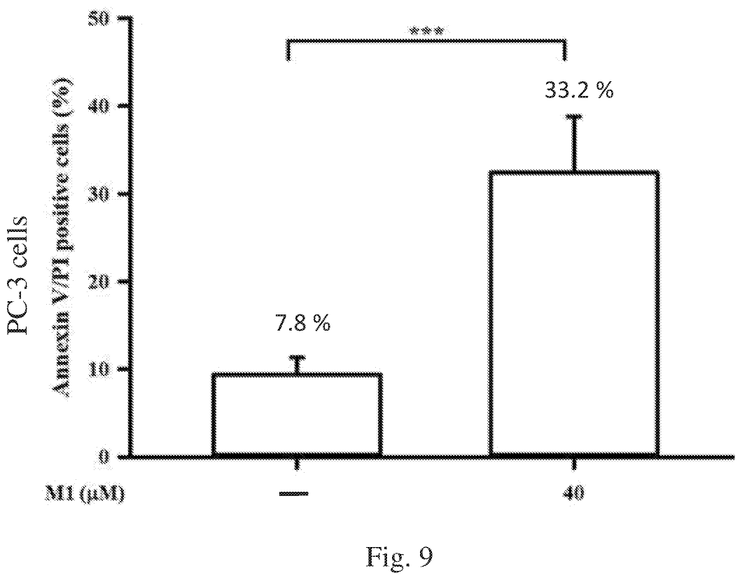

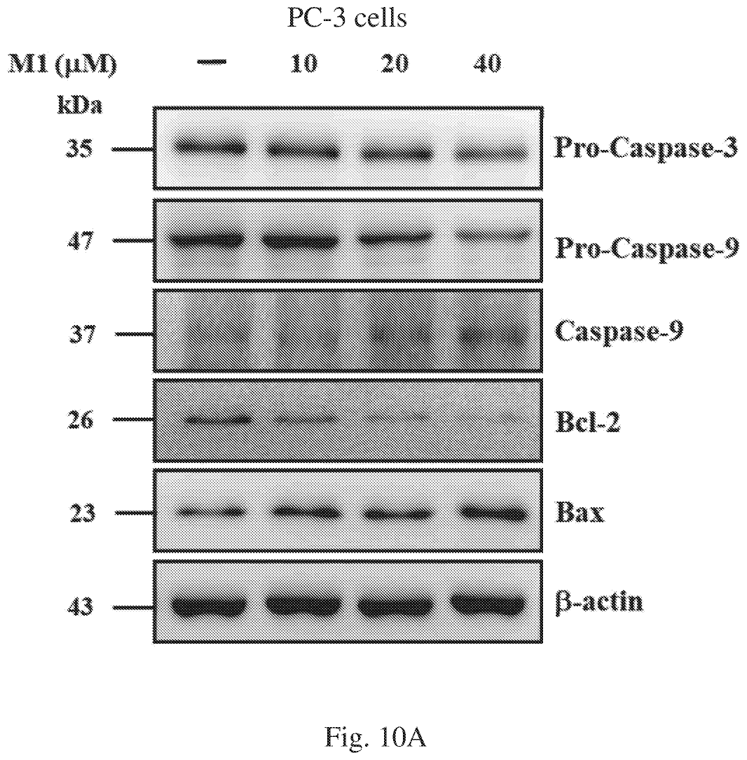

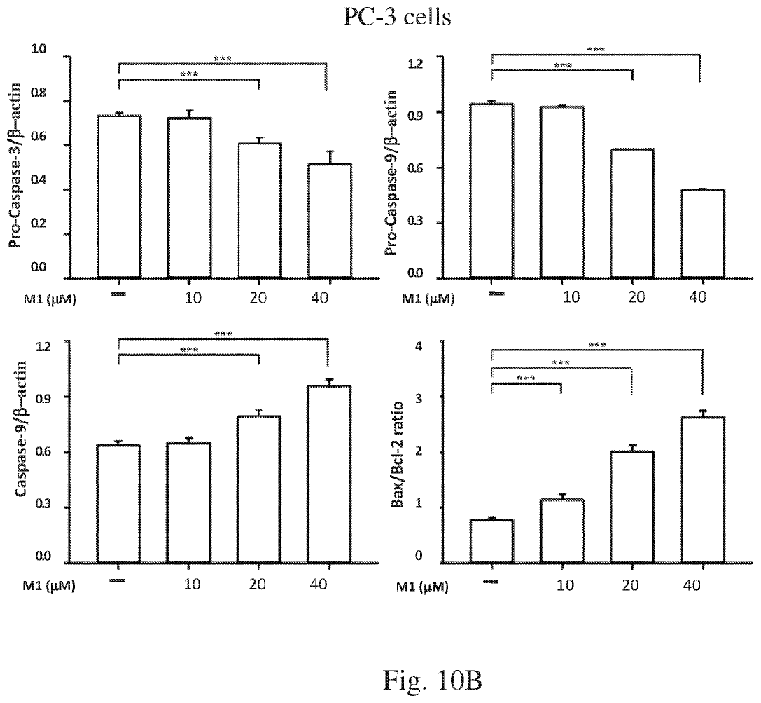

Furthermore, to investigate mechanisms underlying the apoptosis-inducing activity of M1, we detected Annexin V/PI double staining by flow cytometry and apoptosis related protein expression by Western blot. PC-3 cells were plate 2.5×10⁵ per 6-well plates, and incubated with M1 (40 μM) or vehicle for 24 h. The cells were harvested by gently trypsinizing and wash with ice-PBS twice, then stained with Annexin V-FITC/PI apoptosis detection kit (BD Biosciences, USA) according to the manufacturer's instructions and measured using a flow cytometer within 1 hour. As shown in FIG. 9, M1 greatly increased the percentage of PC-3 cells with Annexin V/PI double staining compare to vehicle. Moreover, M1 significantly decreased pro-caspase-3 and pro-caspase-9 protein expression, while increased protein levels of caspase-9 and Bax/Bc1-2 ratio in a dose-dependent manner in PC-3 cells (FIG. 10A and FIG. 10B). These results indicated that M1 induced apoptosis in human prostatic cancer cells. The data are expressed as the means±SEM for three separate experiments. ***p<0.005. (One-way ANOVA and subsequent Scheffe's test).

### REFERENCES

Jia-Fang Liu, Kuang Chi Lai, Shu-Fen Peng, Pornsuda Maraming, Yi-Ping Huang, An-Cheng Huang, Fu-Shin Chueh, Wen-Wen Huang, Jing-Gung Chung. Berberine Inhibits Human Melanoma A375.S2 Cell Migration and Invasion via Affecting the FAK, uPA, and NF-κB Signaling Pathways and Inhibits PLX4032 Resistant A375.S2 Cell Migration In Vitro. Molecules. 2018; 23:2019.
Farid Menaa. Latest Approved Therapies for Metastatic Melanoma: What Comes Next? J Skin Cancer. 2013; 2013:735282.
Nicholas White, Gillian E Knight, Peter E M Butler, Geoffrey Burnstock. An in vivo model of melanoma: treatment with ATP. Purinergic Signal. 2009; 5:327-33.
Megan N Thobe, Robert J Clark, Russell O Bainer, Sandip M Prasad, Carrie W Rinker-Schaeffer. From Prostate to Bone: Key Players in Prostate Cancer Bone Metastasis. Cancers (Basel). 2011; 3:478-93.
Kaustubh Datta, Michael Muders, Heyu Zhang, Donald J Tindall. Mechanism of lymph node metastasis in prostate cancer. Future Oncol. 2010; 6:823-36.

1. A method of treating cancer in a subject in need thereof comprising administering an effective amount of ginsenoside M1 to the subject, wherein the cancer is melanoma or prostate cancer.

2. The method of claim 1, wherein the ginsenoside M1 is administered in an amount effective in inhibiting proliferation, inducing apoptosis, decreasing clonogenic activity and/or reducing migration activity of the cancer cells.

3-7. (canceled)

8. A method for suppressing, reducing, blocking and/or preventing tumor metastasis in a subject in need thereof, comprising administering an effective amount of ginsenoside M1 to the subject, wherein the cancer is melanoma or prostate cancer.

9-11. (canceled)
